(19) United States
(12) Patent Application Publication
(10) Pub. No.: US 2017/0257682 A1
(43) Pub. Date: Sep. 7, 2017

(54) Monitoring Device, System and Method for Monitoring an Individual

(71) Applicant: Yaniv SHTALRYD, Ness Ziona (IL)

(72) Inventor: Yaniv SHTALRYD, Ness Ziona (IL)

(21) Appl. No.: 15/445,144

(22) Filed: Feb. 28, 2017

Related U.S. Application Data
(60) Provisional application No. 62/302,028, filed on Mar. 1, 2016.

(51) Int. Cl. H04Q 9/00; H04L 12/58; G08B 3/10; A61B 5/02; G08B 21/04; A61B 5/00; A61B 5/11; H04W 4/14; G08B 21/06

(52) U.S. Cl. CPC H04Q 9/00; H04W 4/14; H04L 51/04; G08B 3/1008; G08B 21/06; G08B 21/0476; G08B 21/0469; G08B 21/043; A61B 5/447; A61B 5/4818; A61B 5/1113; A61B 5/1117; A61B 5/4094; A61B 5/02; A61B 5/6891

(57) ABSTRACT

A portable monitoring device for monitoring at least one physical parameter of an individual, comprising: a first sensor unit having a senor for sensing the physical parameter of the individual; a control unit configured for receiving data from the first sensor unit and also from at least one other remote second sensor unit, identifying sensing mode of each of the first and second sensor units, and processing the received data; a carrying mechanism for having the portable device carried by the individual to be monitored; and one or more output devices for outputting indication and/or information associated with the processed data. The control unit is configured for processing signal data of the first sensor unit or the remote second sensor unit, according to the identified sensing modes thereof.

### RELATED APPLICATIONS

The present invention claims priority from U.S. provisional patent application No. 62/302,028 filed on Mar. 1, 2016 and incorporated herein by reference.

### FIELD OF THE INVENTION

The present invention generally relates to devices, apparatuses, systems and methods for monitoring one or more primary vital signs for an individual such as, yet not limited to, respiration and/or pulse and the like.

### BACKGROUND OF THE INVENTION

Baby monitoring devices and systems for detection of respiratory failures such as apnea are widespread and typically use piezoelectric transducer sensors embedded in a plate that can be placed under the mattress of the baby bed for sensing respiratory thereof during sleeping. The sensor unit it usually connected via a data communication cable to a control unit which includes a processor for identification of alarming respiratory related situations such as apnea by identifying non-movement of the baby lying over the bed mattress over the sensor unit.

Other monitoring devices and systems are known in the art for monitoring the same and/or other vital signs related parameters such as pulse and/or blood pressure using the same type of sensors or other sensing technologies.

Patent no. EP0514744 by Shtalryd teaches such a system for monitoring respiratory behavior for detecting apnea and outputting an alarm upon detection of breathing cessations using a transducer movement detector.

US patent application publication no. 2008300499 teaches non-invasive monitoring devices for monitoring minute movements made by the body to report the presence or absence of respiration and normal heart rate. The movements being measured are the pulsations made by the normal operation of both the heart and lungs. This is done by converting the body movements into electrical signals with an adapted polymorph-piezoelectric transducer. The electrical signal that is detected is a mixed signal, a signal composed of a lower frequency (breathing contraction) and the higher frequency (heart pulse). The signals are detected by separating the body motions by an adaptive filter that has a break frequency at twice the frequency of the larger body movement signal (breathing contraction). The separate body movement signals operate with associated logic circuitry to allow each signal to be independently measured, recorded, and acted upon to determine if the individual (patient) is undergoing a life threatening experience. Such an event by either signal will trigger an audio and visual display alarm which is attached on or near the patient. The alarm event trigger is also connected to a low voltage detector circuit to indicate that the supply voltage has dropped below an acceptable level.

Patent application no. JPH10295695 teaches a pressure sensing type respiration and body movement sensor, which detests the respiratory movement and the body movement caused by change in pressure applied on the sensor, generally arranged under the breast of a testee in bed. The sound sensor detects the respiration sound such as a snore and is constituted for example, a microphone and a sound conditioner. The recorder which records data obtained from the respiration and body movement sensor and the sound sensor prints or displays the data recorded in a magnetic storage medium or an electric record medium on a display, etc., by a data processing device during or after recording so as to enable the confirmation.

### SUMMARY OF THE INVENTION

It is an object of the present invention to provide systems and methods for monitoring physical conditions.

It is another object of the present invention to provide systems and methods for monitoring physical conditions using a mobile device connected to an external sensor unit.

The present invention thus relates to a monitoring system for monitoring at least one physical condition of an individual, said monitoring system comprising:
a. a sensor unit having at least one sensor for sensing at least one physical parameter of the individual;
b. a mobile device coupled to said sensor unit and configured for:
   i. receiving output signal data from both said sensor unit and from one or more sensors on said mobile device;
   ii. processing data received from both said sensor unit and from said mobile device; and
   iii. outputting an indication and/or information associated with the processed data from both said sensor unit and from said mobile device regarding said at least one physical condition.

In some embodiments, the monitoring system is configured for monitoring at least one of the physical conditions: pressure ulcers, sleep apnea, snoring, wondering, fall prevention, tonic seizures, asthma, sleep tracking, heart conditions, said mobile device further being configured for outputting alarms upon identification of alarming situations.

In some embodiments, the sensor unit comprises a piezoelectric transducer, an infrared sensor or both.

In some embodiments, the sensor unit is configured for being placed and operated over a support of a bed, a crib or other resting furniture upon which the individual is to be rested for monitoring the individual while resting or sleeping thereover.

In some embodiments, the communication between said sensor unit and said mobile device is carried out via a wireless communication link or a wired connection.

In some embodiments, the wireless communication link is based on radio frequency (RF) communication, optical communication or ultrasound communication.

In some embodiments, the wired communication link is a cable connected to the mobile device's headphone jack.

In some embodiments, the mobile device is a mobile phone, a tablet or a portable console.

In some embodiments, the sensors of the mobile device comprise the mobile's device camera and microphone.

In some embodiments, the physical condition is pressure ulcers and the monitoring system uses the sensor's unit piezoelectric transducer and the mobile's device camera to monitor the individual.

In some embodiments, the physical condition is sleep apnea and the monitoring system uses the sensor's unit piezoelectric transducer and the mobile's device microphone to monitor the individual.

In some embodiments, the physical condition is snoring and the monitoring system uses the sensor's unit piezoelectric transducer and the mobile device's microphone to monitor the individual.

In some embodiments, the physical condition is wandering and the monitoring system uses the sensor's unit piezoelectric transducer and infrared sensor, and the mobile device's microphone and camera to monitor the individual.

In some embodiments, the physical condition is fall prevention and the monitoring system uses the sensor's unit piezoelectric transducer and infrared sensor, and the mobile device's microphone to monitor the individual.

In some embodiments, the physical condition is tonic seizures and the monitoring system uses the sensor's unit piezoelectric transducer and the mobile device's microphone and camera to monitor the individual.

In some embodiments, the physical condition is asthma and the monitoring system uses the sensor's unit piezoelectric transducer and the mobile device's microphone to monitor the individual.

In some embodiments, the physical condition is sleep tracking and the monitoring system uses the sensor's unit piezoelectric transducer and the mobile device's camera to monitor the individual.

In another aspect, the present invention relates to a method for monitoring at least one physical condition of an individual, the method comprising:
a. sensing at least one physical parameter of the individual by a sensor unit having at least one sensor;
connecting a mobile device to said sensor unit, said mobile device configured for:
   i. receiving output signal data from both said sensor unit and from one or more sensors on said mobile device;
   ii. processing data received from both said sensor unit and from said mobile phone; and
   iii. outputting an indication and/or information associated with the processed data from both said sensor unit and from said mobile phone regarding said at least one physical condition.

### DETAILED DESCRIPTION OF SOME EMBODIMENTS OF THE INVENTION

In the following detailed description of various embodiments, reference is made to the accompanying drawings that form a part thereof, and in which are shown by way of illustration specific embodiments in which the invention may be practiced. It is understood that other embodiments may be utilized and structural changes may be made without departing from the scope of the present invention.

The present invention provides portable devices, systems and methods using thereof for monitoring at least one physical condition of an individual. The monitoring device comprises a sensor unit connected to a mobile device. The sensor unit has at least one sensor for sensing at least one physical parameter of the individual. The mobile device connected sensor unit is configured for 3 main tasks: i.

receiving output signal data from both the sensor unit and from one or more sensors on the mobile device itself; ii. processing data received from both the sensor unit and from the mobile device sensors; and iii. outputting an indication and/or information associated with the processed data from both the sensor unit and from said mobile device regarding the at least one physical condition.

The monitoring system is configured for monitoring various physical conditions, including but not limited to: pressure ulcers, sleep apnea, snoring, wondering, fall prevention, tonic seizures, asthma, sleep tracking and heart conditions.

The monitoring includes processing data arriving from sensors on both the sensor unit and the mobile device and outputting alarms upon identification of alarming situations. The monitoring system can also output statistics and measured data regarding a physical condition in order to assist both the individual and his caretaker (physician etc.).

The alarms of the monitoring system can be one or more of the following types: alert the person being monitored, for example, issue a sound to wake him up;

alert someone else, like a family member, either living with the monitored individual or not; and alert a caretaker or emergency medical service.

The alerts are typically transmitted via a wireless communication link in a Wifi connection or a mobile data/voice connection (when the mobile device has a SIM card). The alerts messages can be an SMS, an email, a Web message, an instant message or a voice message (prerecorded message and/or using text-to-speech technology). The alerts can also be a sound generated by the mobile device. The mobile device can generate various alerts for the same alarm, for example, call several people and/or send SMS messages to one or more people.

The sensor unit comprises one or more sensors such as piezoelectric crystal sensors (transducers) for sensing minute movements or any other known in the art sensors that can be used for detection/extraction of one or more vital signs related parameters therefrom such as respiration behavior, pulse, blood pressure, sweating, shivering and the like.

The sensor unit is configured for being placed and operated over a support of a bed, a crib or other resting furniture upon which the individual is to be rested for monitoring the individual while resting or sleeping.

The communication between the sensor unit and the mobile device is carried out via a wireless communication link or a wired connection.

The wireless communication link can be based on radio frequency (RF) communication, optical communication or ultrasound communication.

The wired connection is a cable connected to the mobile device's headphone jack.

The mobile device can be a mobile phone, a tablet or a portable console. The mobile device comprises various sensors such as a camera and microphone.

In practice, a user can take a mobile device, connect the mobile device to any external sensor unit, download an application on the mobile device to operate the external sensor and thus use the mobile device as a monitoring platform. By using different external sensor units or by using different application configurations on the mobile device (different applications and/or different modules or options in the same application) the user can adapt the mobile device and sensor units to different kinds of scenarios and needs.

### Examples of Physical Conditions Monitored

Pressure ulcers—the monitoring system uses the sensor's unit piezoelectric transducer and the mobile's device camera to monitor the individual's movements and issue an alert if the individual has not moved or been moved according to the times directed by the medical caretaker. The alert instructs someone in the proximity of the individual (family member, caretaker, medical staff) that it is time to turn the individual.

Sleep apnea—the monitoring system uses the sensor's unit piezoelectric transducer and the mobile's device microphone to monitor the individual to listen to the individual's breath and issue an alert if there is a concern for sleep apnea. The alert can be a sound to wake up the individual and/or a sound or message alert to alert the medical staff or anybody in proximity to the individual.

Snoring—the monitoring system uses the sensor's unit piezoelectric transducer and the mobile device's microphone to listen to the individual breath and identify when snoring occurs. The alert for snoring is typically aimed to wake-up the snoring individual, typically be an audio signal.

Wandering—the monitoring system uses the sensor's unit piezoelectric transducer and infrared sensor, and the mobile device's microphone and camera to monitor the individual's movements and issue an alert if wandering is suspected. The alert is typically to alert someone in proximity for assistance to the individual.

Fall prevention—the monitoring system uses the sensor's unit piezoelectric transducer and/or infrared sensor, and the mobile device's microphone to monitor the individual who is susceptible to falling from bed. The alert is typically to wake-up the individual, usually by audio means.

In some embodiments, issuing an alert to wake-up an individual can also include turning on the light, for example, via the mobile device (as in smart lighting systems that can be operated by a mobile device). In some embodiments, issuing an alert also includes making the mobile device vibrate.

Tonic seizures—the monitoring system uses the sensor's unit piezoelectric transducer and the mobile device's microphone and camera to monitor the individual for tonic seizures. The alert is typically for somebody in the proximity of the individual (family member, caretaker) and/or emergency medical authorities.

Asthma—the monitoring system uses the sensor's unit piezoelectric transducer and the mobile device's microphone to monitor the individual's breath for Asthma attacks.

Sleep tracking—the monitoring system uses the sensor's unit piezoelectric transducer and the mobile device's camera to monitor the individual's sleep patterns and issue statistical information that can help a medical expert in treating sleep disorders of the individual.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following invention and its various embodiments and/or by the following claims. For example, notwithstanding the fact that the elements of a claim are set forth below in a certain combination, it must be expressly understood that the invention includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the invention is explicitly contemplated as within the scope of the invention.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a sub-combination or variation of a sub-combination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptually equivalent, what can be obviously substituted and also what essentially incorporates the essential idea of the invention.

Although the invention has been described in detail, nevertheless changes and modifications, which do not depart from the teachings of the present invention, will be evident to those skilled in the art. Such changes and modifications are deemed to come within the purview of the present invention and the appended claims.

1. A monitoring system for monitoring at least one physical condition of an individual, said monitoring system comprising:
a) a sensor unit having at least one sensor for sensing at least one physical parameter of the individual;
b) a mobile device coupled to said sensor unit and configured for:
i. receiving output signal data from both said sensor unit and from one or more sensors on said mobile device;
ii. processing data received from both said sensor unit and from said mobile device; and
iii. outputting an indication and/or information associated with the processed data from both said sensor unit and from said mobile device regarding said at least one physical condition.

2. The monitoring system according to claim 1, wherein said monitoring system is configured for monitoring at least one of the physical conditions: pressure ulcers, sleep apnea, snoring, wondering, fall prevention, tonic seizures, asthma, sleep tracking, heart conditions, said mobile device further being configured for outputting alarms upon identification of alarming situations.

3. The monitoring system according to claim 1, wherein said sensor unit comprises a piezoelectric transducer, an infrared sensor or both.

4. The monitoring system according to claim 1, wherein said sensor unit is configured for being placed and operated over a support of a bed, a crib or other resting furniture upon which the individual is to be rested for monitoring the individual while resting or sleeping thereover.

5. The monitoring system according claim 1, wherein the communication between said sensor unit and said mobile device is carried out via a wireless communication link or a wired connection.

6. The monitoring system according to claim 5, wherein said wireless communication link is based on radio frequency (RF) communication, optical communication or ultrasound communication.

7. The monitoring system according to claim 5, wherein said wired communication link is a cable connected to the mobile device's headphone jack.

8. The monitoring system according to claim 1, wherein said mobile device is a mobile phone, a tablet or a portable console.

9. The monitoring system according to claim 1, wherein the sensors of the mobile device comprise the mobile's device camera and microphone.

10. The monitoring system according to claim 1, wherein said physical condition is pressure ulcers and the monitoring system uses the sensor's unit piezoelectric transducer and the mobile's device camera to monitor the individual.

11. The monitoring system according to claim 1, wherein said physical condition is sleep apnea and the monitoring system uses the sensor's unit piezoelectric transducer and the mobile's device microphone to monitor the individual.

12. The monitoring system according to claim 1, wherein said physical condition is snoring and the monitoring system uses the sensor's unit piezoelectric transducer and the mobile device's microphone to monitor the individual.

13. The monitoring system according to claim 1, wherein said physical condition is wandering and the monitoring system uses the sensor's unit piezoelectric transducer and infrared sensor, and the mobile device's microphone and camera to monitor the individual.

14. The monitoring system according to claim 1, wherein said physical condition is fall prevention and the monitoring system uses the sensor's unit piezoelectric transducer and infrared sensor, and the mobile device's microphone to monitor the individual.

15. The monitoring system according to claim 1, wherein said physical condition is tonic seizures and the monitoring system uses the sensor's unit piezoelectric transducer and the mobile device's microphone and camera to monitor the individual.

16. The monitoring system according to claim 1, wherein said physical condition is asthma and the monitoring system uses the sensor's unit piezoelectric transducer and the mobile device's microphone to monitor the individual.

17. The monitoring system according to claim 1, wherein said physical condition is sleep tracking and the monitoring system uses the sensor's unit piezoelectric transducer and the mobile device's camera to monitor the individual.

18. A method for monitoring at least one physical condition of an individual, the method comprising:
a) sensing at least one physical parameter of the individual by a sensor unit having at least one sensor;
b) connecting a mobile device to said sensor unit, said mobile device configured for:
i. receiving output signal data from both said sensor unit and from one or more sensors on said mobile device;
ii. processing data received from both said sensor unit and from said mobile phone; and
iii. outputting an indication and/or information associated with the processed data from both said sensor unit and from said mobile phone regarding said at least one physical condition.
